(19) United States
(12) Patent Application Publication
(10) Pub. No.: US 2019/0134231 A1
(43) Pub. Date: May 9, 2019

(54) METHODS AND COMPOSITIONS FOR DETECTING ANEURYSMS

(71) Applicant: THE BOARD OF REGENTS OF THE UNIVERSITY OF TEXAS SYSTEM, Austin, TX (US)

(72) Inventors: Reto ASMIS, San Antonio, TX (US); Sina TAVALOKI, San Antonio, TX (US)

(73) Assignee: THE BOARD OF REGENTS OF THE UNIVERSITY OF TEXAS SYSTEM, Austin, TX (US)

(21) Appl. No.: 16/095,155

(22) PCT Filed: Apr. 19, 2017
(86) PCT No.: PCT/US17/28280

§ 371 Date: Oct. 19, 2018

Related U.S. Application Data
(60) Provisional application No. 62/325,811, filed on Apr. 21, 2016.

(51) Int. Cl. A61K 49/00; A61K 51/08; A61K 45/06; A61K 9/00; A61K 31/663

(52) U.S. Cl. CPC A61K 49/0056; A61K 51/088; A61K 45/06; A61K 49/0043; A61K 31/663; A61K 49/0041; A61K 9/0019

(57) ABSTRACT

Provided herein are methods and compositions for the detection of vulnerable, rupture-prone aneurysms using multi-meric folate conjugated peptides (FCPs). Further provided are methods for treating aneurysms identified to be at risk of rupture.

This application claims benefit of priority to U.S. Provisional Application Ser. No. 62/325,811, filed Apr. 21, 2016, the entire contents of which are hereby incorporated by reference.

The invention was made with government support under Grant No. UL1TR001120 awarded by the National Institutes of Health. The government has certain rights in the invention.

This invention was made with government support under grant no. TR001120 awarded by the National Institutes of Health. The government has certain rights in the invention.

### BACKGROUND OF THE INVENTION

### 1. Field of the Invention

The present invention relates generally to the fields of chemistry and medicine. More particularly, it concerns methods and compositions for the detection of aneurysms.

### 2. Description of Related Art

Abdominal aortic aneurysm (AA) is one of the most common vascular diseases, particularly in the elderly male population, affecting about 1.1 million patients in the US (Hirsch et al., 2006 and Kent et al., 2010). Its prevalence increases from 1-2% in people 45-54 years old to 12.5% in males and 5.2% in females at the 75-84 years of age (Kuivaniemi et al., 2008, Hirsch et al., 2006, Singh et al., 2001 and Powell and Greenhalgh, 2003). Not surprisingly, both the incidence and mortality attributed to AA have continued to rise with the aging of the population in developed countries (Klink et al., 2011, Wilmink et al., 1999, Lederle et al., 2000, Upchurch and Schaub, 2006 and Norman and Powell, 2007). While the majority of AA remains stable or demonstrate slow expansion over years, they may enlarge or eventually rupture in a subset of patients (Guirguis and Barber, 1991). The resulting exsanguination is associated with a high mortality even in the minority of patients who survive long enough to receive advanced critical care and emergent vascular repair, with a periprocedural mortality rate >50% (Peppelenbosch et al., 2003, Kuivaniemi et al., 2008, Buxton 2012, Thomas and Stewart, 1988, Farooq et al., 1996 and Ernst 1993). Elective vascular repair, as the definitive treatment of AA, carries a considerable risk of peri-procedural mortality (3-5% for surgical and 0.5-2% for endovascular repair) (Brady et al., 2000, Dillavou et al., 2006 and Lee et al., 2004) in addition to the risk of debilitating complications, e.g., ischemic renal injury (Kudo et al., 2004), spinal cord ischemia and paraplegia (Peppelenbosch et al., 2005). Considering that the majority of AA never ruptures and the co-morbid conditions of this aged population, preventive vascular repair imposes an unnecessary risk to patients which may outweigh its benefit, and is estimated to cause 1400 deaths in the US annually (Aggarwal et al., 2011 and McPhee et al., 2007). However, conventional imaging modalities are limited in prediction of AA outcome beyond the determination of the aneurysm size. Thus, molecular imaging of the biological processes associated with progression of AA may play a critical role in clinical decision making and improve the risk stratification of patients (Tavakoli 2009 and Libby et al., 2010).

Macrophages are among the most abundant inflammatory cells in the aneurysmal vessel wall and are a major source of extracellular proteases including matrix metalloproteinases (MMPs) which reduce the tensile strength of the vessel wall allowing for arterial expansion under the influence of mechanical forces (Razavian et al., 2010, Nahrendorf et al., 2011, El-Hamamsy and Yacoub, 2009 and Davies 1998). The critical role of monocyte-derived macrophages in development and progression of AA has been shown by suppression of AA expansion with various approaches that deplete them from the vessel wall or diminish their recruitment (Kanematsu et al., 2011, de Waard et al., 2010 and Moehle et al., 2011). The abundance and pivotal role of macrophages in the pathogenesis of AA make them attractive targets for molecular imaging (Razavian et al., 2010 and Nahrendorf et al., 2011). Various approaches have been employed in pre-clinical and small clinical studies with promising prospects to detect macrophage activation in AA, including detection of enhanced metabolic (Courtois et al., 2013, Marini et al., 2012 and Kotze et al., 2011) or phagocytic (Nahrendorf et al., 2011, Yao et al., 2012 and Richards et al., 2011) activity, and targeting cell surface proteins (Kitagawa et al., 2013 and Kitagawa et al., 2012) or secreted proteolytic enzymes (Razavian et al., 2010 and Golestani et al., 2015). However, the small size of the vessel wall and limitations of the current tracers (including lack or limited cell specificity (e.g., ¹⁸F-FDG), limited depth of penetration into the lesions (e.g. nanoparticles), and insufficient affinity/uptake of the tracers to achieve in vivo detectability) are challenges to their successful translation into clinical application. Accordingly, there is a need to develop a non-invasive imaging modality for detection of ongoing recruitment and differentiation of monocyte-derived macrophages which provides a venue for prospective prediction of AA outcome.

### SUMMARY OF THE INVENTION

Embodiments of the present disclosure provide methods and compositions for the detection and/or treatment of aneurysms. In a first embodiment, there is provided a multimeric compound comprising at least two folate conjugated peptides (FCPs), wherein a FCP comprises a folate receptor beta (FRβ) ligand and a detectable label. In certain aspects, the FRβ ligand is pteroyl-γ-glutamate or an analog thereof.

In some aspects, a first FCP of the at least two FCPs is identical to a second FCP. In other aspects, a first FCP of the at least two FCPs is not identical to a second FCP. In some aspects, the second FCP comprises a different FRβ ligand and/or detectable label as compared to the first FCP.

In certain aspects, the detectable label comprises a chromophore. In some aspects, the chromophore is a fluorophore. For example, the fluorophore is cyanine, fluorescein, rhodamine, DyLight Fluor or Alexa Flour. In particular aspects, the rhodamine is tetramethylrhodamine (TAMRA).

In some aspects, the detectable label is a radionuclide. In certain aspects, the radionuclide is a positron-emitting isotope or a gamma-ray isotope. In particular aspects, the gamma-ray isotope is ^{99m}Tc. In certain aspects, the radionuclide further comprises a chelating crosslinker. For example, the chelating crosslinker is hydrazinonicotinamide (HyNic), diethylene triamine pentaacetic acid (DTPA), or 1,4,7,10-tetraazacyclododecane-1,4,7,10-tetraacetic acid (DOTA). In particular aspects, the chelating crosslinker is hydrazinonicotinamide (HyNic)

In some aspects, the detectable label is conjugated to the FRβ ligand by a spacer. In certain aspects, the spacer comprises the amino acid sequence ARSK. In particular aspects, the spacer is conjugated to the γ-glutamate of the pteroyl-γ-glutamate.

In certain aspects, the at least two FCPs further comprise a second detectable label. In some aspects, the first detectable label comprises a chromophore and the second detectable label comprises a radionuclide. In other aspects, the first detectable label comprises a radionuclide and the second detectable label comprises a chromophore.

In some aspects, the at least two FCPs are conjugated by a linker. In certain aspects, the linker is cleavable by a matrix metalloproteinase (MMP) or a cathepsin. In some aspects, the MMP is further defined as MMP2/9. In particular aspects, the MMP2/9-cleavable linker comprises an amino acid sequence GPLGLAGRP.

In certain aspects, the first FCP comprises from N-terminus to C-terminus the pteroyl-γ-glutamate; a first spacer; TAMRA; a second spacer; HyNic-^{99m}Tc and the MMP2/9 cleavable linker.

In some aspects, the multiemric compound further comprises a therapeutic agent. In certain aspects, the therapeutic agent is a protein, a peptide, or a therapeutic nucleic acid. In some aspects, the therapeutic nucleic acid is an antisense, a siRNA, an antisense, or a gene therapy. In certain aspects, the protein is an antibody, and antibody fragment, an scFv, or an antigen. In particular aspects, the therapeutic agent is an anti-aneurysmal agent. For example, the anti-aneurysmal agent is bisphosphonate, angiotensin-converting enzyme inhibitor, beta-blocker, or statin.

In another embodiment, there is provided a pharmaceutical composition comprising a multimeric compound of the embodiments, a therapeutic agent and an excipient. In some aspects, the pharmaceutical composition is formulated for oral, intravenous, intraarticular, parenteral, enteral, topical, subcutaneous, intramuscular, buccal, sublingual, rectal, intravaginal, intrapenile, intraocular, epidural, intracranial, or inhalational administration.

In yet another embodiment, there is provided a method of treating an aneurysm in a subject comprising administering a therapeutically effective amount of the pharmaceutical composition of the embodiments to said subject. In some aspects, the aneurysm is an aortic aneurysm or a cerebral aneurysm.

A further embodiment provides a method for producing a multimeric compound comprising at least two FCPs, wherein an FCP comprises pteroyl-γ-glutamate, TAMRA, HyNic-^{99m}Tc, and a MMP2/9 cleavable linker, comprising: (a) combining N¹⁰-(Trifluoroacetyl)pteroic acid and N-α-Fmoc-L-glutamic acid α-t-butyl ester, thereby producing pteroyl-γ-glutamate; (b) adding Fmoc-Lysine(5-TAMRA)-OH and Fmoc-Lysine-ε-(6-Boc-HyNic); (c) attaching the MMP2/9 cleavable linker, thereby producing an FCP; and (d) conjugating the least two FCPs through the MMP2/9 cleavable linker, thereby producing the multimeric compound.

In an even further embodiment, there is provided a method for detecting a rupture-prone aneurysm comprising administering an effective amount of the multimeric compound of the embodiments to a subject and imaging the detectable moiety, wherein focal uptake of the multimeric compound identifies a rupture-prone aneurysm. In some aspects, the subject is a human In some aspects, the subject has a previously diagnosed aneurysm.

In certain aspects, the imaging comprises positron emission tomography (PET), single photon emission computed tomography (SPECT), computerized tomography (CT), or magnetic resonance imaging (MRI). In some aspects, the imaging is performed by a catheter-based device.

In certain aspects, the aneurysm is an aortic aneurysm or cerebral aneurysm. In some aspects, the aortic aneurysm is an abdominal aortic aneurysm or a thoracic aortic aneurysm. In other aspects, the cerebral aneurysm is a saccular aneurysm or a fusiform aneurysm.

In some aspects, administering comprises injection. In certain aspects, the injection is intraperioneal, intravenous or intramuscular. In some aspects, administering is performed at least 30 minutes prior to imaging.

In certain aspects, the MMP2/9 cleavable linker is cleaved at site of a vulnerable aneurysm. In some aspects, the multimeric compound has enhanced penetration into the vessel wall after cleavage by MMP2/9 relative to prior to cleavage. In some aspects, the multimeric compound selectively binds small peritoneal macrophages as compared to large peritoneal macrophages.

Another embodiment provides a method of treating a rupture-prone aneurysm comprising performing surgical repair and/or administering an anti-aneurysmal agent to the subject identified to have the rupture-prone aneurysm according to the embodiments. In some aspects, an anti-aneurysmal agent is bisphosphonate, angiotensin-converting enzyme inhibitor, beta-blocker, or statin. In certain aspects, treating comprises inhibiting the development of or inducing the regression of the aneurysm. In some aspects, surgical repair comprises placing a stent graft.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C: Schematic structure of (FIG. 1A) exemplary FCPmon, (FIG. 1B) exemplary FCPtet, and (FIG. 1C) exemplary FCPoct.

FIGS. 2A-B: FRβ is expressed at low levels by only about 5% of steady state peritoneal F4/80bright LPM (FIG. 2A). However, the majority of thioglycollate-elicited F4/80int SPM (FIG. 2B) express FRβ at markedly higher levels. The smaller size and lower expression of F4/80 signal by SPM (FIG. 2B) compared to the LPM (FIG. 2A) can be noted. FRβ: Red. F4/80: Green.

FIGS. 3A-C: Flow cytometry (FIG. 3A) demonstrates specific uptake of FCPmon (red) by thioglycollate elicited peritoneal monocytes-macrophages (F4/80⁺, CD115⁺) (FIG. 3B), but not by other elicited cells (F4/80⁻, CD115⁻) (FIG. 3C). FCPmon specificity is confirmed by inhibition of the uptake in the presence of 100 molar excess of non-labeled folic acid (green) (B&C).

FIGS. 4A-D: Five days post-thioglycollate injection, peritoneal monocytes and macrophages were analyzed by flow cytometry after exclusion of debris (FIG. 4A) and CD115⁻/F4.80⁻cells (FIG. 4B). Monocytes, SPM and LPM were gated based on the expression level of F4/80 (FIG. 4C). Despite the lack of FCPmon uptake in recently recruited monocytes, high level of uptake was noted after differentiation to SPM, which was subsequently downregulated upon maturation to LPM (FIG. 4D).

FIG. 5: LPS and IL-4 loading of Matrigel plugs skew the recently recruited macrophages into classical proinflammatory and alternative antiinflammatory activation states, respectively, as evidenced by expression of established markers Nos2 and Fizz1l. However, Folr2 (gene encoding FR-β) expression is independent of the macrophage activation state.

FIGS. 6A-B: Mean fluorescent intensity (MFI) analysis (FIG. 6A) confirms the concentration dependent FCPmon uptake by thioglycollate elicited macrophages in the nM concentration range (1.6 to 100 nM). (FIG. 6B) Overlay FACS histograms and MFI bar charts demonstrates 3.8-fold increased FCPmon uptake after incubation at 37° C. compared to 4° C. Near complete inhibition of uptake by excess non-labeled folic acid confirms the specificity of FCPmon uptake. Incubation with BSA has no effect on FCPmon uptake.

FIGS. 7A-F: Images from control (FIG. 7A) and inflamed (FIG. 7B) muscles 1 week after injection of turpentine oil demonstrates myositis and angiogenesis in the inflamed leg. CD11b⁺/Ly6G⁻ monocytes-macrophages and CD11b⁺/Ly6G⁺ neutrophils are gated on flow cytometry analysis of inflamed muscles 1 week after oil injection (FIG. 7C). FCPmonuptake is only identified in monocyte-macrophage, but not neutrophil, gate (FIG. 7D). FCPmon uptake is primarily identified in cells which have lost their Ly6C expression (FIG. 7E), suggesting that FRβ expression occurs after differentiation of monocytes to macrophages. An example negative control sample is represented in (FIG. 7F).

FIGS. 8A-D: Axial (FIG. 8A), coronal (FIG. 8B) and sagittal (FIG. 8C) [CT (top row), microSPECT (middle row) and fused microSPECT/CT (bottom row)] as well as the volume rendered (FIG. 8D) images 1 week after injection of turpentine oil into the thigh demonstrate focal uptake of FCPmon. Red asterisks indicate the hypoattenuating turpentine oil collection at the injection site which is surrounded by intense uptake of FCPmon.

FIGS. 9A-G: Examples of axial CT angiogram (FIG. 9A), microSPECT (FIG. 9B) and fused microSPECT/CT (FIG. 9C) obtained 1-hour after intravenous injection of 0.5 mCi of FCPmon demonstrate focal uptake in the aneurysmal segment of the aorta (red circle). Inferior vena cava (blue circle) is obscured by scattered radiation from the kidney. Ex vivo photographs and corresponding planar imaging of the excised aortas confirm the focal uptake of FCPmon in the aneurysmal (red arrows, FIGS. 9E & 9G) compared to non-aneurysmal (FIGS. 9D & 9F) segments.

FIGS. 10A-D: Atherosclerotic aortas from hyperlipidemic mice (N=3) were dissociated into single cells and analyzed by FACS. Macrophages are gated by inclusion of CD11b⁺ myeloid cells after exclusion of dead cells (DAPI), neutrophils (Ly6G⁺) (FIG. 10A) and monocytes (Ly6C⁺) (FIG. 10B). About 5.8%±1.0% of Mac3⁻ and 5.0%±0.8% of Mac3⁺ macrophages internalize FCPmon (FIG. 10C). FRβ specificity of FCPmon uptake is confirmed by negative staining (FIG. 10D) and higher RNA expression of FRβ in sorted FCPmon-positive cells compared to FCPmon-negative cells.

### DESCRIPTION OF ILLUSTRATIVE EMBODIMENTS

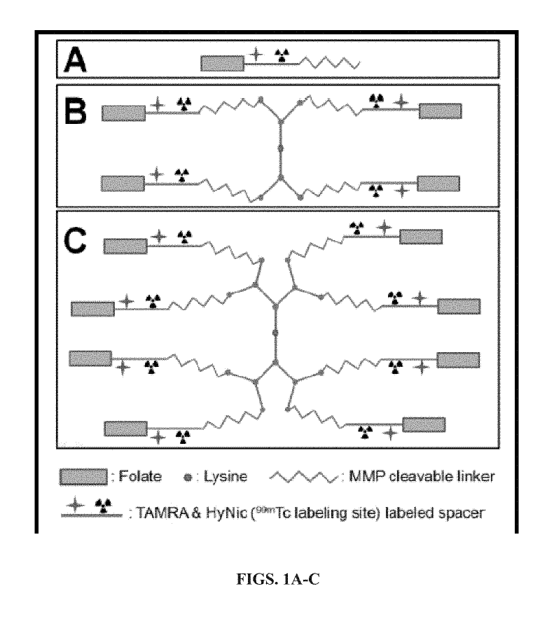

Rupture of aortic aneurysm (AA) causes about 15,000 deaths in the US annually (Kent et al., 2004, Ince and Nienaber, 2007, Peppelenbosch et al., 2003; Kuivaniemi et al., 2008). Current clinical and conventional imaging criteria for identification of aneurysms at “high risk” of rupture are suboptimal (Razavian et al., 2010; Golestani et al., 2015). Accordingly, the present disclosure provides methods and compositions for the detection of rupture-prone aneurysms. Specifically, the inventors have synthesized a novel folate conjugated peptide (FCP) and established its specificity and high affinity to FRβ expressing macrophages. In some aspects, the FCP is fluorescent- and/or radio-labeled for detection by imaging. An additional feature of FCP is the incorporation of a matrix metalloproteinase (MMP)-2/9 cleavable linker at its C-terminus which allows developing multimeric FCPs, such as tetra (FCPₜₑₜ) and octameric (FCP_{oct}) tracers (FIGS. 1B & C), which upon cleavage release multiple radiolabeled targeting small peptides. This strategy takes advantage of the increased avidity of the multimeric tracers and the enhanced penetration of the cleaved small peptides through the vessel wall at sites of high MMP activity which may result in specific signal amplification in aneurysmal vessel wall. Thus, the FRβ-targeting peptides provided herein have high efficiency and with dual modality labeling, allowing the supplementation of the scintillation-based imaging with high resolution fluorescence microscopy. Accordingly, the present disclosure provides highly sensitive non-invasive methods for detecting and subsequently treating vulnerable aneurysms.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

As used herein, the term “subject” refers to an animal, particularly a mammal, even more particularly a human. The terms “patient” and “subject” are used interchangeably herein. The conditions diagnosed, monitored and/or treated by means of the present disclosure occur primarily in mammalian subjects. Human patients are by far the most important subjects treatable according to the method of the disclosure, but the method can be practiced for the benefit of other mammals, including, for example, pet animals such as dogs and cats, laboratory animals such as rats and mice, as well as farm animals such as horses and cows.

“Therapeutically effective amount” means the amount of a compound that, when administered to a subject for treating a disease or disorder, is sufficient to effect such treatment for the disease or disorder. The “therapeutically effective amount” can vary depending on the compound, the disease or disorder and its severity, and the age or weight of the subject to be treated.

“Treating” or “treatment” of any disease or disorder refers, in one embodiment, to ameliorating the disease or disorder (i.e., arresting or reducing the development of the disorder or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder, or even preventing the same (i.e., a prophylactic therapy).

As used herein, the term “aneurysm” refers to a localized, blood-filled dilation (balloon-like bulge) of a blood vessel caused by disease or weakening of the vessel wall. Aneurysms most commonly occur in arteries at the base of the brain (the circle of Willis) and in the aorta (the main artery coming out of the heart, an aortic aneurysm). As the size of an aneurysm increases, there is an increased risk of rupture, which can result in severe hemorrhage, other complications or even death. “Aneurysm” further means not only conventional vascular aneurysms, but also refers to any abnormal localized dilatations of blood vessels. A “rupture-prone” or “vulnerable” aneurysm refers to an aneurysm at increased risk for rupture.

“Abdominal aortic aneurysm” (also known as “AAA” or abdominal “AA”) is a localized dilatation (ballooning) of the abdominal aorta exceeding the normal diameter by more than 50 percent. Approximately 90 percent of abdominal aortic aneurysms occur infrarenally (below the kidneys), but they can also occur pararenally (at the level of the kidneys) or suprarenally (above the kidneys). Such aneurysms can extend to include one or both of the iliac arteries in the pelvis. Abdominal aortic aneurysms occur most commonly in individuals between 65 and 75 years old and are more common among men and smokers. They tend to cause no symptoms, although occasionally they cause pain in the abdomen and back (due to pressure on surrounding tissues) or in the legs (due to disturbed blood flow). The major complication of abdominal aortic aneurysms is rupture, which can be life-threatening as large amounts of blood spill into the abdominal cavity, and can lead to death within minutes.

“Chelating agent” refers to a molecule, often an organic one, having two or more unshared electron pairs available for donation to a metal ion, whereby such complexes involving the bound metal ion includes two or more atoms of the chelant.

“Moiety” refers a specific segment or functional group of a molecule. Chemical moieties are often recognized chemical entities embedded in or appended to a molecule.

“Peptide” and “polypeptide” are used interchangeably and refer to a polymer of amino acids. A “polypeptide” is a polymer of amino acid residues joined by peptide bonds, whether produced naturally or synthetically. Polypeptides of less than about 10 amino acid residues are commonly referred to as “peptides.”

“Radioisotope” or “radionuclide” refer to atoms having an unstable nucleus (characterized by excess energy) available to be imparted to a newly-created radiation particle within the nucleus. The radioisotope undergoes radioactive decay emitting gamma rays.

### II. ANEURYSMS

Embodiments of the present disclosure provide methods and compositions for the detection of aneurysms that are at risk for rupture. Aneurysms are a complex multifactorial disease with genetic and environmental risk factors. Genetic factors have been shown to play a role in the etiology of aneurysms even when they are not associated with Marfan syndrome,

Ehlers-Danlos syndrome, Loeys-Dietz syndrome, or other rare aortic syndromes. Since aneurysms result from the weakening, rather than the hardening, of arteries, it is not yet clear whether calcification is physiologically relevant to the formation and progression of aneurysms. Though it was once assumed that aneurysms occurred as a consequence of advanced atherosclerosis, increasing evidence suggests that they may be distinct phenomena that share a few, but not all, clinical risk factors (Golledge et al., 2010). Whether this association between aneurysms and atherosclerosis is causal or simply due to common risk factors is unknown.

Aneurysm size is one of the strongest predictors of the risk of rupture, with risk increasing markedly at aneurysm diameters of greater than 5.5 cm (Aggarwal et al., 2011). The five-year overall cumulative rupture rate of incidentally diagnosed aneurysms in population-based samples is 25% to 40% for aneurysms larger than 5.0 cm, compared with 1% to 7% for aneurysms 4.0 cm to 5.0 cm in diameter (Nevitt et al., 1989). A statement from the Joint Council of the American Association for Vascular Surgery and Society for Vascular Surgery (Brewster et al., 2003) estimated the annual rupture risk according to AAA diameter to be the following:
Less than 4.0 cm in diameter—0%
4.0 cm to 4.9 cm in diameter—0.5% to 5%
5.0 cm to 5.9 cm in diameter—3% to 15%
6.0 cm to 6.9 cm in diameter—10% to 20%
7.0 cm to 7.9 cm in diameter—20% to 40%
8.0 cm in diameter or greater—30% to 50%

The expansion rate may also be an important determinant of the risk of rupture (Gadowski et al., 1994). A small AAA that expands 0.5 cm or more over six months of follow-up is considered to be at high risk for rupture (Hirsch et al., 2006). Growth tends to be more rapid in smokers, and less rapid in patients with diabetes mellitus or peripheral vascular disease. In addition to aneurysm size and expansion rate, other factors that increase the risks of rupture are continued smoking, uncontrolled hypertension and increased wall stress.

A. Aortic Aneurysms

The risk of abdominal aortic aneurysms (AAAs) increases dramatically in the presence of the following factors: age older than 60 years, smoking, hypertension and Caucasian ethnicity (Aggarwal et al., 2011). The majority of AAAs are asymptomatic and are detected as an incidental finding on ultrasonography, abdominal computed tomography or magnetic resonance imaging performed for other purposes. It can also present with abdominal pain or complications such as thrombosis, embolization and rupture. Approximately 30% of asymptomatic AAAs are discovered as a pulsatile abdominal mass on routine physical examination. Abdominal ultrasonography is considered the screening modality of choice for detecting AAAs because of its high sensitivity and specificity, as well as its safety and relatively lower cost. Management options for patients with an asymptomatic AAA include reduction of risk factors such as smoking, hypertension and dyslipidemia; medical therapy with beta-blockers; watchful waiting; endovascular stenting; and surgical repair depending on the size and expansion rate of the aneurysm and underlying comorbidities.

The risk of AAAs increases dramatically after 60 years of age (Singh et al., 2001). Clinically relevant aneurysms (more than 4 cm in diameter) are present in approximately 1% of men between 55 and 64 years of age, and the prevalence increases by 2% to 4% per decade thereafter (Singh K et al., 2001). AAAs are four to six times more common in men than in women (Scott et al., 1995). In addition, AAAs develop in women approximately 10 years later than in men.

B. Cerebral Aneurysms

Intracranial aneurysms are present in roughly 5% of the population, yet most are often asymptomatic and never detected (Seibert et al., 2011). Development of an aneurysm typically occurs during adulthood, while formation and growth are associated with risk factors such as age, hypertension, pre-existing familial conditions, and smoking. Subarachnoid hemorrhage, the most common presentation due to aneurysm rupture, represents a serious medical condition often leading to severe neurological deficit or death. Recent technological advances in imaging modalities, along with increased understanding of natural history and prevalence of aneurysms, have increased detection of asymptomatic unruptured intracranial aneurysms (UIA). Studies reporting on the risk of rupture and outcomes have provided much insight, but the debate remains of how and when unruptured aneurysms should be managed.

Treatment methods include two major intervention options: clipping of the aneurysm and endovascular methods such as coiling, stent-assisted coiling, and flow diversion stents. The risks associated with endovascular repair are lower and incur shorter hospital stays for appropriately selected patients. The endovascular treatment option should be considered based on factors such as aneurysm size, location, patient medical history, and operator experience.

There are two main types of brain aneurysms—saccular (berry) aneurysms and fusiform aneurysms. The most common type of aneurysm is saccular. Saccular or berry aneurysms look like a sack and are usually formed at the bifurcation or “Y” formation when a larger vessel splits into two vessels. These types of aneurysms are often found at the branches of larger arteries at the base of brain but may be found in other areas of the brain, too.

The second type, a fusiform aneurysm, is less common than a saccular aneurysm and is more stable and seldom ruptures. Fusiform aneurysms occur at the junction of the “Y” formation where a blood vessel branches and extends into both smaller vessels and also into the single larger vessel. Fusiform aneurysms do not develop any stems like saccular aneurysms.

C. Diagnosis

AAA is usually diagnosed by physical exam, ultrasound, or computerized tomography (CT). Alternative less often used methods for visualization of an aneurysm include magnetic resonance imaging (MRI) and angiography. An asymptomatic AAA is often discovered incidentally because of the performance of abdominal USG, CT or magnetic resonance imaging for other purposes. An AAA may also be found with plain x-rays showing some calcification in the wall of the aneurysm. However, they are not reliable because some aneurysms do not have sufficient calcification to be detected. The diagnosis of an AAA should ideally be made before the development of clinical symptoms to prevent rupture. Accordingly, the present disclosure provides methods and compositions for non-invasively detecting aneurysms, particularly rupture-prone aneurysms.

Large aneurysms in thin people are easy to detect. The accuracy of the clinical examination is tremendously reduced by obese body habitus and small aneurysm size (Chervu et al., 1995). However, the physical examination has considerably variable interobserver sensitivity for detection of AAAs. The sensitivity of physical examination for the identification of an AAA ranges from 22% to 96%, and even an experienced physician may miss palpating an AAA in the presence of obesity or abdominal distention (US Preventive Services Task Force Guide to Clinical Preventive Services, 1996).

D. Methods of Treatment

In some embodiments, the present disclosure provides methods for the treatment of aneurysms once they have been detected to be vulnerable to rupture. The methods of treatment include one or more of the anti-aneurysmal agents known in the art and/or surgical repair.

The current treatment options for asymptomatic aneurysms, such as AAA, are conservative management, surveillance with a view to eventual repair, and immediate repair. There are currently two modes of repair available for an AAA: open aneurysm repair (OR), and endovascular aneurysm repair (EVAR). An intervention is often recommended if the aneurysm grows more than 1 cm per year or it is bigger than 5.5 cm. Repair is also indicated for symptomatic aneurysms. Open techniques include bypass surgery with a prosthetic graft and excision. Bypass surgery of an aneurysm means placing the prosthetic graft to cut off blood flow through the aneurysm. If the aneurysm is infected or mycotic, it may then be excised (i.e., cut out and removed from the body). If uninfected, the aneurysm is often left in place.

Conservative management is indicated in patients where repair carries a high risk of mortality and in patients where repair is unlikely to improve life expectancy. The mainstay of the conservative treatment is smoking cessation. Surveillance is indicated in small asymptomatic aneurysms (less than 5.5 cm) where the risk of repair exceeds the risk of rupture. As an AAA grows in diameter the risk of rupture increases. Surveillance until the aneurysm has reached a diameter of 5.5 cm has not been shown to have a higher risk as compared to early intervention.

For aneurysms in the aorta, arms, legs, or head, the weakened section of the vessel may be replaced by a bypass graft that is sutured at the vascular stumps. Instead of sewing, the graft tube ends, made rigid and expandable by nitinol wireframe, can be inserted into the vascular stumps and permanently fixed there by external ligature. New devices were recently developed to substitute the external ligature by expandable ring allowing use in acute ascending aorta dissection, providing airtight, easy and quick anastomosis extended to the arch concavity. Less invasive endovascular techniques allow covered metallic stent grafts to be inserted through the arteries of the leg and deployed across the aneurysm.

Although the data regarding therapeutic benefit of beta-blockers in management of AAA are limited, beta-blockers have been shown to significantly reduce the expansion rate of AAA when monitored by serial USG examination (Gadowski et al., 1994). The 2005 ACC/AHA guidelines recommended beta-blocker therapy in patients with an AAA who do not undergo surgery. Because of the possible attenuation of aneurysm expansion, beta-blockers are also a preferred drug for patients with hypertension or angina with care taken in patients with atrioventricular blocks, bradycardia, chronic obstructive pulmonary disease and peripheral vascular disease.

Interest in antibiotic therapy in the management of AAA is based on evidence of chronic inflammation in AAA, inhibition of proteases and inflammation by antibiotics, and possible involvement of Chlamydia pneumoniae in the pathogenesis of AAA. A study (Vammen et al., 2001) evaluating the role of antibiotics in the management of AAA found a reduction in the mean annual expansion rate of the aneurysms among patients receiving an antibiotic (roxithromycin) compared with those receiving placebo therapy. Also, long-term use of antibiotics has been associated with an increased risk for breast cancer. With uncertain benefits and known harms, more reassuring data are needed before this approach can be recommended.

The beneficial role of treating cardiovascular risk factors, such as hypertension and dyslipidemia, in aneurysm formation, growth or rupture are not clear. However, these approaches may prolong survival by their effect on cardiac and cerebrovascular disease. Long-term statin use has been found to reduce all-cause mortality in patients who underwent previous successful surgical repair of an AAA. The 2005 ACC/AHA guidelines recommended that patients with AAAs should have their blood pressure and lipids controlled as recommended for patients with atherosclerotic disease. A retrospective study concluded that statins may also be of therapeutic benefit in patients who are treated medically, reducing mortality and possibly slowing growth of the aneurysm.

### III. METHODS OF DETECTION

Embodiments of the present disclosure provide methods and compositions for the detection, targeting and/or treatment of aneurysms, particularly rupture-prone aneurysms, through folate conjugated peptides (FCP). The FCP can recognize rupture-prone aneurysms by recognition of recently recruited and differentiated macrophages at the site of the aneurysms by selectively binding to folate receptor beta (FRβ). In some aspects, the FCP comprises a FRβ and a detectable moiety, optionally conjugated by a linker. In certain aspects, multimeric compounds are provided herein in which at least two FCPs are conjugated. The FCP subunits of the multimeric compounds may or may not be identical. In some aspects, the FCPs have different FRβ ligands and/or different detectable moieties.

A. Folate Receptor Ligand

In some embodiments, the FCP comprises an FRβ ligand which binds to FRβ. There are two major isoforms of the human membrane folate binding proteins, α and β. The two isoforms have ^{˜}70% amino acid sequence homology, and differ dramatically in their stereospecificity for some folates. Both isoforms are expressed in both fetal and adult tissue; normal tissue generally expresses low to moderate amounts of FR-β. A ligand of FRβ is folic acid or pteroyl glutamic acid which is a vitamin consisting of a pteridine ring linked by a methylene bridge to a para-aminobenzoic acid moiety, which is joined through an amide linkage to a glutamic acid residue. In some aspects, the FRβ ligand is folic acid (i.e., pteroyl-γ-glutamate) with the following formula:

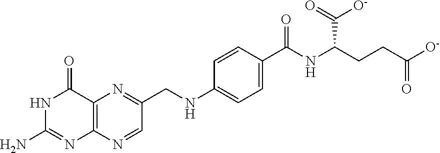 [SMILES: Nc1nc2ncc(CNc3ccc(C(=O)N[C@@H](CCC(=O)[O-])C(=O)[O-])cc3)nc2c(=O)[nH]1]

In some aspects, the FRβ comprises an analog or derivate of folic acid, such as an analog with increased selectivity for FRft Analogs and/or derivatives of folic acid include folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate. The dideaza analogs include, for example, 1,5-dideaza, 5,10-dideaza, 8,10-dideaza, and 5,8-dideaza analogs of folate. Other folate receptor-binding analogs include aminopterin, amethopterin (methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). Additional analogs of folic acid that bind to folic acid receptors are described in U.S. Patent Application Publication Nos. 2005/0227985 and 2004/0242582, the disclosures of which are incorporated herein by reference.

B. Detectable Moieties

The FCP further comprises at least one detectable moiety such that the compound can be detected by imaging. For example, the detectable moiety can be a chromophore or radionuclide. In particular aspects, the FCP comprises two detectable labels, such as a chromophore and a radionuclide with or without a linker. In some aspects, one FCP of a multimeric compound may comprise a first detectable label and the second FCP may comprise a second detectable label. Alternatively, the first FCP may have two detectable labels while the second FCP only has one detectable label.

1. Chromophore

In some aspects, the detectable moiety is a chromophore. Chromophores are molecules capable of selective light absorption resulting in the coloration of these molecule containing compounds. The color arises when a molecule at an excited state releases energy in the form of light with a defined spectrum. Exemplary chromophores include, but are not limited to, a fluorochrome, a non-fluoro chrome chromophore, a quencher (e.g. fluorescence quencher and a dark quencher), an absorption chromophore, a fluorophore, any organic or inorganic dye, metal chelate, or any fluorescent enzyme substrate. In some aspects, the chromophore is a fluorochrome. In some aspects, the fluorochrome is a fluorophore. In some aspects, the chromophore is a quencher. In some aspects, the chromophore is a dark quencher. The FCPs in the multimeric compounds provided in the present disclosure may contain different fluorophores and quenchers, such as for FRET assays.

Several chromophores are described in the art, e.g. Beriraan, Handbook of Fluorescence Sprectra of Aromatic Molecules, 2nd Edition, Academic Press, New York, (1971). In examples that utilize fluorescent labels as described herein, any suitable fluorescent label may be used. Exemplary fluorophores suitable for use with the present disclosure includes rhodamine, rhodol, fluorescein, thiofluorescein, aminofiuorescein, carboxyfiuorescein, chlorofluorescein, methylfluorescein, sulfofiuorescein, aminorhodol, carboxyrhodol, chlororhodol, methylrhodol, sulforhodol; aminorhodamine, carboxyrhodamine, chlororhodamine, methylrhodamine, sulforhodamine, and thiorhodamine; cyanine, indocarbocyanine, oxacarbocyanine, thiacarbocyanine, merocyanine, cyanine 2, cyanine 3, cyanine 3.5, cyanine 5, cyanine 5.5, cyanine 7, oxadiazole derivatives, pyridyloxazole, nitrobenzoxadiazole, benzoxadiazole, pyren derivatives, cascade blue, oxazine derivatives, Nile red, Nile blue, cresyl violet, oxazine 170, acridine derivatives, pro flavin, acridine orange, acridine yellow, arylmethine derivatives, auramine, crystal violet, malachite green, tetrapyrrole derivatives, porphin, phtalocyanine and bilirubin; 1-dimethylaminonaphthyl-5-sulfonate, 1-anilino-8-naphthalene sulfonate, 2-p-touidinyl-6-naphthalene sulfonate, 3-phenyl-7-isocyanatocoumarin, N-(p-(2-benzoxazolyl)phenyl)maleimide, stilbenes, pyrenes, 6-FAM (Fluorescein), 6-FAM (NHS Ester), Fluorescein dT, HEX, JOE (NHS Ester), MAX, TET, ROX, TAMRA, TARMA™ (NHS Ester), TEX 615, ATTO™ 488, ATTOTM 532, ATTO™ 550, ATTO™ 565, ATTO™ Rho1O1, ATTO™ 590, ATTO™ 633, ATTO™ 647N, TYE™ 563, TYE™ 665, and TYE™ 705. In particular aspects, the chromophore is TAMRA.

2. Radionuclide

In some aspects, the detectable moiety is a radionuclide. Suitable radionuclide labels are Tc, In, Ga, Cu, F, Lu, Y, Bi, Ac, and other radionuclide isotopes. Particularly, the radionuclide is selected from the group comprising ¹¹¹In, ^{99m}Tc, ^{94m}Tc, ⁶⁷Ga, ⁶⁶Ga, ⁶⁸Ga, ⁵²Fe, ⁶⁹Er, ⁷²As, ⁹⁷Ru, ²⁰³Pb, ₆₂Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁵⁷Gd, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{177m}Sn, ²¹³Bi, ¹⁴²Pr, ¹⁴³Pr, ¹⁹⁸Au, ¹⁹⁹Au, ¹⁸F, ¹²³I, ¹²⁴I, ¹³¹I, ⁷⁵Br, ⁷⁶Br, ⁷⁷Br, and ⁸²Br, amongst others. These radionuclides are cationic and can be complexed with the chelator through the chelating group of the conjugate to form labeled FCP.

Two radionuclides commonly used in nuclear imaging and suitable for the present embodiments are the positron emitter ¹⁸F (used in PET), and the gamma ray emitter ^{99m}Tc (used in SPECT). These radionuclides have relatively short half-lives (109 minutes and 6 hours, respectively) that make them favorable for minimizing exposure of the body to radiation, and have decay characteristics that make them optimal for their respective imaging modalities. However, focusing on PET imaging, the relatively short half-life of ¹⁸F and its typical labeling conditions (use of organic solvents) lowers its suitability for use with biomolecules such as antibodies. An alternative radionuclide may be the positron emitter ⁶⁴Cu²⁺.

In particular aspects, the radionuclide is Technetium-99m, a metastable nuclear isomer of technetium-99 and is symbolized as ^{99m}Tc. The “m” indicates that it is a metastable nuclear isomer, which means that it does not change into another element (i.e., transmutate) upon a decay. It is a gamma ray emitting isotope used in radioactive isotope medical tests, for example as a radioactive tracer that medical equipment can detect in the body. It is well suited to the role because it emits readily detectable 140 keV gamma rays, and its half-life for gamma emission is 6.01 hours (meaning that about fifteen sixteenths (93.7%) of it decays to ^{99m}Tc in 24 hours). The short half-life of the isotope allows for scanning procedures which collect data rapidly, but keep total patient radiation exposure low.

The radioisotope may attached to the FRβ ligand by a chelation moiety that is covalently or non-covalently bonded to the FRβ ligand and is chelated with the radioisotope, or is part of a chemical intermediate that is covalently or non-covalently bonded to the polypeptide. Chelation is the binding or complexation of a bi- or multidentate ligand. These ligands, which are often organic compounds, are called chelants, chelators, chelating agents or sequestering agent. Chelants, according to ASTM-A-380, are “chemicals that form soluble, complex molecules with certain metal ions, inactivating the ions so that they cannot normally react with other elements or ions to produce precipitates or scale.” The ligand forms a chelate complex with the substrate. The term is reserved for complexes in which the metal ion is bound to two or more atoms of the chelant. Exemplary radionuclides and/or chelation moieties are described in U.S. Pat. No. 8,778,303.

In certain embodiments, the radioisotope is a technetium core, and the chelation moiety includes one or more of a natural peptide, such as Gly-Ser-Cys, Gly-Gly-Cys, Cys-Gly-Cys, Lys-Gly-Cys, Gly-Ala-Gly, His-His-His; or a chelation structure that contains single or multiple atoms selected from one or more of nitrogen, sulfur, and/or oxygen, such as structures configured as N₂S_{4,} N₂S_{3,} N₂S_{2,} N₃S; or a modified peptide, such as a mercaptoacetyltriglycine (MAG₃), a MAG₂-NH_{2,} a Benzoyl-MAG₃, and a Methyl-MAG₂-NH₂. In some such embodiments, the compound may also include one or more coligands which participate in the chelation structure. Suitable coligands include tricine, phosphine compounds, dicine, bicine, glucoheptonate, ethylenediamine-N,N′-diacetate (EDDA), imine-N-heterocycle, and pyridine-2-azo-p-dimethylaniline (PADA).

In embodiments where the radioisotope is ^{99m}Tc, chelation moieties include one or more of a hydrazine, a diazenido, a diazene, an isodiazene, a hydrazinopyrimidine, a hydrazone, a hydrazinonicotinamide (HyNic), and 2-hydrazinopyridine. Such embodiments may further include one or more coligands selected from tricine, a glucoheptonate, ethylenediamine-N,N′-diacetate (EDDA), dicine, bicine, an imine-N-heterocycle, pyridine-2-azo-p-diamethylaniline (PADA), and a phosphine derivative. In particular aspects, the chelation moiety is HyNiC.

In embodiments where the radioisotope is ¹¹¹In^{3+, 111}In^{0, 111}In^{5-,201}Tl, ¹⁷⁷Lu, ⁶⁴Cu^{+, 64}Cu²⁺, or ⁶⁴Cu³⁺, chelation moieties include one or more of ethylenediaminetetraacetic acid (EDTA), diethylenetriamine pentaacetic acid (DTPA), and tetraazacyclododecanetetraacetic acid (DOTA). In embodiments where the radioisotope is ¹⁸⁶Re or ¹⁸⁸Re, chelation moieties include one or more of a monoamidemonoaminedithiol (MAMA) and a MAG₃. In embodiments where the radioisotope is ⁶⁷Ga or ⁶⁸Ga, a chelation moiety is a triazacyclononanetriacetic acid (NOTA). In embodiments where the radioisotope is ¹²⁵I⁻, ¹²⁵I⁰, ¹²⁵I⁷⁺, ¹³¹I⁻, ¹³¹I⁰, or ¹³¹I⁷⁺, chemical intermediates include one or more of a tyrosyl moiety and a hydrazone. Alternatively, the iodine isotope may be incorporated into the peptide without the involvement of a chemical intermediate.

C. Linkers

The compounds of present disclosure may optionally include a linker, spacer, or couple of variable length. The linker, spacer, or couple, hereinafter collectively referred to as a “linker,” is adapted for connecting the FRβ to another molecule in other embodiments of the disclosure, such as a detectable moiety, or for connecting one FCP to another FCP. Such linkers are known in the art and are often used to “associate” one chemical entity to another. As used herein, the term “association” refers to any manner of coexistence of two or more molecules, such as complexation, chelation, ion-pairing, covalant bonding, and the like, such that for a time sufficient to administer the associated molecules, the associated molecules may be interpreted as a single entity.

The linker may create either a permanent or a semipermanent (i.e., labile) linkage. Semi-permanent linkers may depend upon endogenous mechanisms of cleavage, and include metabolically labile linkers, such as a nucleotide, amide, or an ester, subject to cleavage by peptidases, esterases, phosphodiesterases, and reductases, which provides a stable ligand-agent conjugate prior to delivery but allows cleavage upon reaching the target or treatment site. For example, the linker may be cleavable by a protease or cathepsin.

In some aspects, the linker is a peptide between about 2 to about 20 amino acids. A peptide linker may be of any suitable length, such as, for example, about 3 to about 30, or particularly about 6 to about 24 atoms in sequence (e.g., a linear peptide about 1 to 10 or particularly about 2 to 8 amino acids long). For example, a peptide linker may comprise the sequence ARSK.

The linker may be cleavable under physiological conditions, such as by a protease. A cleavable peptide linker may include an amino acid sequence recognized and cleaved by a protease, so that proteolytic action of the protease cleaves the linker. Exemplary linkers are described in U.S. Patent Application No. 2007/0041904, U.S. Pat. Nos. 8,664,407, and 8,399,403, all incorporated herein by reference.

One important class of signals is the hydrolytic activity of matrix metalloproteinases (MMPs), which are very important in the invasive migration of metastatic tumor cells. MMPs are also believed to play major roles in inflammation and stroke. MMPs are reviewed in Visse et al. (2003). MMPs may be used to cleave a linker and allow a multimeric FCP compound to release monomeric FCPs. For example, a linker may include the amino-acid sequence PLGLAG that is cleaved by the metalloproteinase enzyme MMP-2 or GPLGLAGRP that is cleaved by MMP-2/9.

D. Imaging

In certain embodiments, this disclosure contemplates methods of imaging aneurysms using FCPs (e.g., monomeric or multimeric). The FCP can be labeled with fluorescence and/or radioactivity which can be detected by various methods known in the art.

Nuclear Magnetic Resonance (NMR) and Magnetic Resonance Imaging (MRI) are techniques for identifying isotopes in a sample (area) by subjecting the sample to an external magnetic fields and detecting the resonance frequencies of the nuclei. An MRI scanner typically consists of magnet of 1.5 to 7, or more Tesla strength. A magnetic field and radio waves are used to excite protons in the body. These protons relax after excitation, and a computer program translates this data into pictures of human tissue. In certain embodiments, this disclosure contemplates that a pre-contrast image is taken. Once the FCPs are injected, a post-contrast image is taken. A contrast is detected wherever the FCPs aggregate in the body.

NMR typically involves the steps of alignment (polarization) of the magnetic nuclear spins in an applied, constant magnetic field and perturbation of this alignment of the nuclear spins by employing an electro-magnetic radiation, usually radio frequency (RF) pulse. A pulse of a given carrier frequency contains a range of frequencies centered about the carrier frequency. The Fourier transform of an approximately square wave contains contributions from the frequencies in the neighborhood of the principal frequency. The range of the NMR frequencies allows one to use millisecond to microsecond radio frequency pulses.

Single-photon emission computed tomography (SPECT) is an imaging technique using gamma rays. Using a gamma camera, detection information is typically presented as cross-sectional slices and can be reformatted or manipulated as required. One injects a gamma-emitting radioisotope (radionuclide) into a subject. The radioisotope contains or is conjugated to a molecule that has desirable properties, e.g., a marker radioisotope has been attached to a ligand, folate. This allows the combination of ligand, e.g., folate, and radioisotope (the radiopharmaceutical) to be carried and bound to a place of interest in the body, which then (due to the gamma-emission of the isotope) allows the ligand concentration to be seen by a gamma-camera.

Positron emission tomography (PET) is an imaging technique that produces a three-dimensional image. The system detects pairs of gamma rays emitted indirectly by a positron-emitting radionuclide (tracer). Three-dimensional images of tracer concentration within the area are then constructed by computer analysis. A radioactive tracer isotope is injected into subject, e.g., into blood circulation. Typically there is a waiting period while tracer becomes concentrated in tissues of interest; then the subject is placed in the imaging scanner. As the radioisotope undergoes positron emission decay, it emits a positron, an antiparticle of the electron with opposite charge, until it decelerates to a point where it can interact with an electron, producing a pair of (gamma) photons moving in approximately opposite directions.

These are detected in the scanning device. The technique depends on simultaneous or coincident detection of the pair of photons moving in approximately opposite direction (the scanner has a built-in slight direction-error tolerance). Photons that do not arrive in pairs (i.e. within a timing-window) are ignored. One localizes the source of the photons along a straight line of coincidence (also called the line of response, or LOR). This data is used to generate an image.

Light having a wavelength range from 600 nm and 850 nm lies within the near infrared range of the spectrum, in contrast to visible light, which lies within the range from about 400 nm to about 500 nm. Therefore, the excitation light used in practice of the disclosure diagnostic methods will contain at least one wavelength of light to illuminates the tissue at the infrared wavelength to excite the compounds in order that the fluorescence obtained from the area having uptake of the compounds of the present disclosure is clearly visible and distinct from the auto-fluorescence of the surrounding tissue. The excitation light may be monochromatic or polychromatic. In this manner, the compounds of the present disclosure are advantageous as they eliminate the need for use of filtering mechanisms that would be used to obtain a desired diagnostic image if the fluorescent probe is one that fluoresces at wavelengths below about 600 nm. In this manner, the compounds of the present disclosure avoid obscured diagnostic images that are produced as a result of excitation light of wavelengths that would be reflected from healthy tissue and cause loss of resolution of the fluorescent image.

Diagnostic labs, physicians' offices and operating rooms for surgical procedures can be equipped with an overhead light that produces wavelengths of light in the optical emitting spectrum useful in practice of disclosure diagnostic methods, such as lamps that produce light in the appropriate wavelength. Such a light can be utilized in the practice of the disclosure diagnostic methods merely by turning out the other lights in the operating room (to eliminate extraneous light that would be visibly reflected from tissue in the body part under investigation) and shining the excitation light of near infrared wavelength into the body cavity or surgically created opening so that the fluorescent image received directly by the eye of the observer (e.g., the surgeon) is predominantly the fluorescent image emanating from the fluorophore(s) in the field of vision.

Within any of the imaging embodiments, methods disclosed herein may further comprise the steps of recording the images from an area of the subject on a computer or computer readable medium. In certain embodiments, the methods may further comprise transferring the recorded images to a medical professional representing the subject under evaluation.

In some aspects, the compounds of the present disclosure are used to identify a aneurysm by administering such compounds for a time and under conditions that allow for binding of the compound to at least one cell of the target cell type (e.g., recently recruited and differentiated macrophages). The bound compound is then optically detected such that presence of fluorescence of the near infrared wavelength emanating from the bound, targeted compound of the present disclosure indicated that the target cell type is present in the biological sample.

The amount of the conjugate compound effective for use in accordance with the method of the disclosure depends on many parameters, including the molecular weight of the conjugate, its route of administration, and its tissue distribution. In accordance with the disclosure an “effective amount” of the FCP conjugate is an amount sufficient to bind to activated macrophages and to be useful in the identification/monitoring of rupture-prone aneurysms. The effective amount of the ligand conjugate to be administered to a patient being evaluated for anerysms can range from about 1 ng/kg to about 10 mg/kg, or from about 10 μg/kg to about 1 mg/kg, or from about 100 μg/kg to about 500 μg/kg.

The FCP compound can be administered in one or more doses (e.g., about 1 to about 3 doses) prior to the catheterization or external imaging procedure. The number of doses depends on the molecular weight of the compound, its route of administration, and its tissue distribution, among other factors. When used for identification/monitoring of rupture-prone aneurysms, the catheterization or external imaging procedure is typically performed about 1 to about 6 hours post-administration of the FCP compound targeted to recently differentiated macrophages, but the catheterization or external imaging procedure can be performed at any time post-administration of the FCP compound as long as binding of the FCP to recently differentiated macrophages is detectable.

The FCP compounds administered in accordance with the method of the present disclosure may be administered parenterally to the patient being evaluated for aneurysms, for example, intravenously, intradermally, subcutaneously, intramuscularly, or intraperitoneally, in combination with a pharmaceutically acceptable carrier. Suitable means for parenteral administration include needle (including microneedle) injectors, needle-free injectors and infusion techniques. Alternatively, the FCP compounds can be administered to the patient being evaluated for aneurysms by other medically useful procedures such as in an orally available formulation. In accordance with the disclosure, a “patient being evaluated for aneurysms” means any patient suspected of having aneurysms, whether symptomatic or not, who would benefit from an evaluation using the method of the present disclosure.

E. Targeted Delivery of Anti-Aneurysmal Agents

In some embodiments, methods are provided for the use of the FCP of the present disclosure for the targeted delivery of a therapeutic agent, such as an anti-aneurysmal agent, to the site of an aneurysm. The FCP or the multimeric compound of FCPs can be conjugated to a therapeutic agent and administered to a subject in an amount effective to treat the aneurysm. Examples of anti-aneurysmal agents that may be conjugated to one or more FCPs include a beta-blocker, an antibody (e.g., roxithromycin), bisphosphonate, angiotensin-converting enzyme inhibitor, or statin. Other therapeutic agents that may be conjugated to the FCP include a protein, siRNA, small molecule or nanoparticle. In some aspects, the FCP may be conjugated to more than one therapeutic agent. The FCP-drug conjugate may also be administered in combination with surgical repair. The FCP-drug conjugate may be administered prior to, simultaneously with or after the surgical repair to treat the aneurysm.

Therapeutic agents may be bound to the FCP of the present disclosure by known methods in the art (e.g., by covalent bond, noncovalent interactions, or expressed as a fusion or chimeric protein). A therapeutic agent or multiple therapeutic agents may be bound to a carrier, as well as multiple types of therapeutic agents. In a further embodiment, a therapeutic agent may be bound to a carrier using a linker. For example, (BIOCONJUGATE TECHNIQUES, 1996) describes techniques for modifying or crosslinking of biomolecules. For example, a diagnostic agent and a pharmaceutically active agent may be bound to a FCP of the present disclosure. In another example, multiple types of agents may be bound to a carrier, such as at least one pharmaceutically active agent, at least one biologic agent, at least one diagnostic agent and at least one targeting agent, or various combinations thereof.

In some aspects, the present disclosure provides a pharmaceutical composition comprising the FCPs of the present disclosure and a therapeutic agent, and may include a pharmaceutically acceptable carrier, suitable for administration to a mammal, particularly a human. To administer the pharmaceutical composition to humans or animals, it is preferable to formulate the molecules in a composition comprising one or more pharmaceutically acceptable carriers. The phrase “pharmaceutically or pharmacologically acceptable” refers to molecular entities and compositions that do not produce allergic, or other adverse reactions when administered using routes well-known in the art. “Pharmaceutically acceptable carriers” include any and all clinically useful solvents, dispersion media, coatings, antibacterial and antifungal agents, isotonic and absorption delaying agents and the like.

The compounds of the disclosure may be incorporated into convenient dosage forms, such as capsules, impregnated wafers, tablets or particularly, injectable preparations. Solid or liquid pharmaceutically acceptable carriers may be employed.

Solid carriers include starch, lactose, calcium sulfate dihydrate, terra alba, sucrose, talc, gelatin, agar, pectin, acacia, magnesium stearate and stearic acid. Liquid carriers include syrup, peanut oil, olive oil, saline, water, dextrose, glycerol and the like. Similarly, the carrier or diluent may include any prolonged release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., a solution), such as an ampoule, or an aqueous or nonaqueous liquid suspension. A summary of such pharmaceutical compositions may be found, for example, in Gennaro, 2005.

The pharmaceutical preparations are made following conventional techniques of pharmaceutical chemistry involving such steps as mixing, granulating and compressing, when necessary for tablet forms, or mixing, filling and dissolving the ingredients, as appropriate, to give the desired products for oral, parenteral, topical, transdermal, intravaginal, intrapenile, intranasal, intrabronchial, intracranial, intraocular, intraaural and rectal administration. The pharmaceutical compositions may also contain minor amounts of nontoxic auxiliary substances such as wetting or emulsifying agents, pH buffering agents and so forth.

Examples of pharmaceutically acceptable carriers or additives include water, a pharmaceutical acceptable organic solvent, collagen, polyvinyl alcohol, polyvinyl-pyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present disclosure.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis and Characterization of Folate Conjugated Peptides

Using a solid phase peptide synthesizer and reverse-phase high-performance liquid chromatography a HyNic and fluorochrome (carboxytetramethylrhodamine: TAMRA) labeled monomeric peptide (FCPₘₒₙ) was synthesized and purified. FCPₘₒₙ (FIG. 1) is composed of 1) N-terminal folic acid (pteroyl-γ-glutamate): constituting the FRβ targeting moiety conjugated to the peptide through the γ-carboxyl group of glutamate (to retain the affinity to FRβ) using commercially available reagents [N₁₀-(Trifluoroacetyl)pteroic acid (Sigma) and N-α-Fmoc-L-glutamic acid α-t-butyl ester (EMD Millipore)] (Lee and Low, 2000 and Zhang et al., 2004); 2) TAMRA and HyNic tagged spacer [Ala-Arg-Ser-Lys(TAMRA)-Ala-Arg-Ser-Lys(HyNic)-Ala]: allowing for high efficiency and site specific fluorescent and 99m-Tc labeling using commercially available modified lysine residues [Fmoc-Lysine(5-TAMRA)-OH (AAT Bioquest) and Fmoc-Lysine-ε-(6-Boc-HyNic)-OH (Solulink)]; and 3) C-terminal MMP-2/9-cleavable linker (Gly-Pro-Leu-Gly˜Leu-Ala-Gly-Arg-Pro) (Jiang et al., 2004) which allows for cleavage of the labeled peptides from the multimeric tracers at sites of proteolytic activity.

To evaluate the relationship between FRβ expression and monocyte recruitment, the peritoneal compartment was studied before the complex model of vessel wall inflammation. Monocytes recruited to inflamed peritoneum go through multiple maturation stages, which are identifiable by expression of surface markers such as CD-115 (Csfr1 or M-CSF receptor) and F4/80. These subsets include: a) CD115⁺/F4.80^{dim} newly recruited and yet undifferentiated monocytes; b) CD115⁺/F4.80^{int} recently differentiated small peritoneal macrophages (SPM); and eventually c) CD115⁺/F4.80^{bright} mature large peritoneal macrophages (LPM) (Ghosn et al., 2010 and Nguyen et al., 2012). In uninflamed peritoneum, LPMs comprise more than 90% of macrophages; but LPM disappear from peritoneum within hours after an inflammatory insult through migration to omentum and regional lymph nodes. This is followed by influx of monocytes which then differentiate into SPMs within the first few days of inflammation and subsequently reconstitute the LPMs during the resolution phase of inflammation (Ghosn et al., 2010 and Nguyen et al., 2012). This predictable pattern facilitates evaluation of the temporal relationship between FRβ expression and monocyte flux and differentiation.

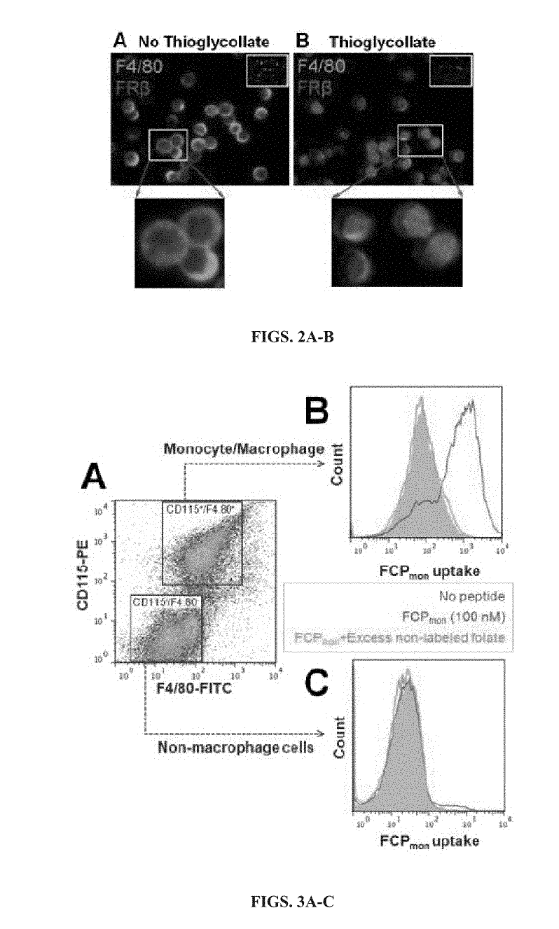

Sterile peritonitis was introduced by intra-peritoneal injection of thioglycollate in C57BL/6 mice and the elicited cells were harvested at day +3 (Xia et al., 2009) Immunostaining with anti-FRβ (Pierce) and anti-F4/80 (Serotec) antibodies demonstrated strong FRβ staining by the vast majority of the recently recruited F4/80^{int} SPM (FIG. 2); whereas, FRβ signal was detectable on only a small subset, and at a significantly lower level, of F4/80^{bright} LPM in the steady state (FIG. 2). Using fluorescence-activated cell sorting (FACS), it was demonstrated that the monomeric peptide, FCPₘₒₙ, specifically binds to and is internalized by activated and recently recruited SPM, and not by other thioglycollate elicited peritoneal cells, e.g., neutrophils, eosinophils and lymphocytes (FIG. 3). Consistent with the immunostaining data, about 5% of LPM internalized FCPₘₒₙ under steady state condition, albeit at a lower capacity compared to thioglycollate-elicited macrophages.

To confirm that uptake of FCPₘₒₙ is attributable to FRβ, the mRNA expression was measured in sorted peritoneal macrophages which showed 19.1-fold higher expression of FRβ mRNA in FCPₘₒₙ-positive compared to FCPₘₒₙ-negative macrophages. Interestingly, FACS analysis during the mid-phase of peritonitis when LMP reappear (5-6 days after thioglycollate injection) demonstrated different levels of FCPmon uptake by the monocytes-macrophage subsets (FIG. 4): a) monocytes showed no significant FCPₘₒₙ uptake; while b) SPM had the highest uptake; and c) LPM showed an intermediate uptake of FCPₘₒₙ. These data are consistent with the early and transient expression of FRβ during the early phase of monocyte differentiation; suggesting that non-invasive imaging of FRβ allows detection of the ongoing flux and differentiation of monocyte-derived macrophages in inflammatory foci.

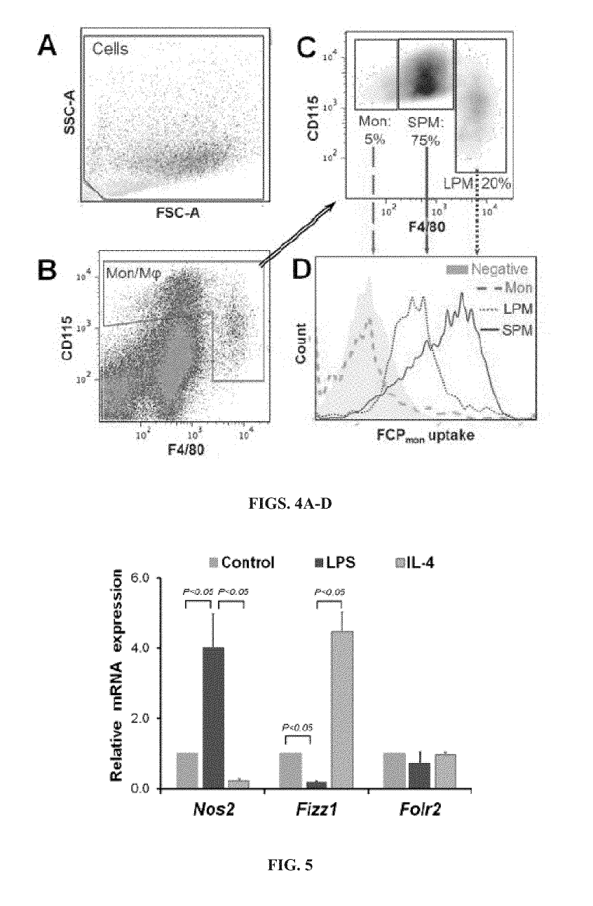

The pro-versus anti-inflammatory activation state of FRβ expressing macrophages is controversial. Initially identified in active inflammatory foci (such as peritonitis and rheumatoid arthritis synovium), these cells were favored to represent proinflammatory or classically activated macrophages (Xia et al., 2009, van der Heij den et al., 2009, Ayala-Lopez et al., 2010, Lu et al., 2011, Feng et al., 2011, Varghese et al., 2007, Paulos et al., 2006, Paulos et al., 2004 and Turk et al., 2002). However, other studies have reported that FRβ co-localizes with markers of anti-inflammatory activation in lung (Shen et al., 2013), atherosclerotic plaque (Jager et al., 2014) and tumor associated (Puig-Kroger et al., 2009, Nagai et al., 2009 and Hattori et al., 2014) macrophages. To address if FRβ expression is affected by the activation states, C57BL/6 mice were subcutaneously injected with Matrigels plugs (BD biosciences) loaded with either lipopolysaccharide (LPS) or recombinant murine IL-4 to produce pro- and anti-inflammatory skewed microenvironments, respectively. The pro-and anti-inflammatory activation states of the isolated cells were confirmed by overexpression of Nos2 and Fizz1, respectively (FIG. 5). However, FRβ expression was independent of the activation state in this in vivo model (FIG. 5).

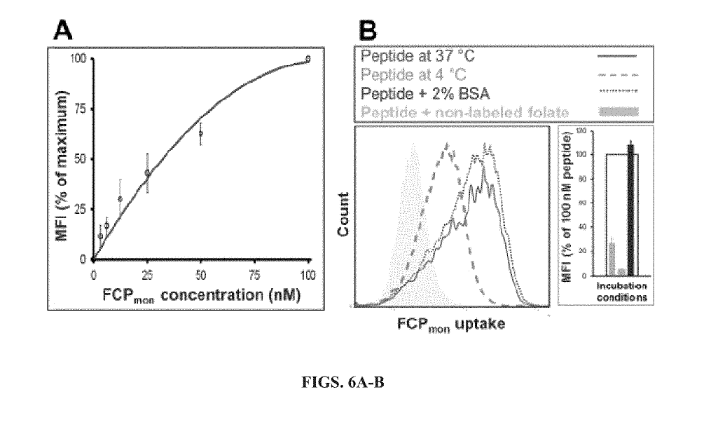

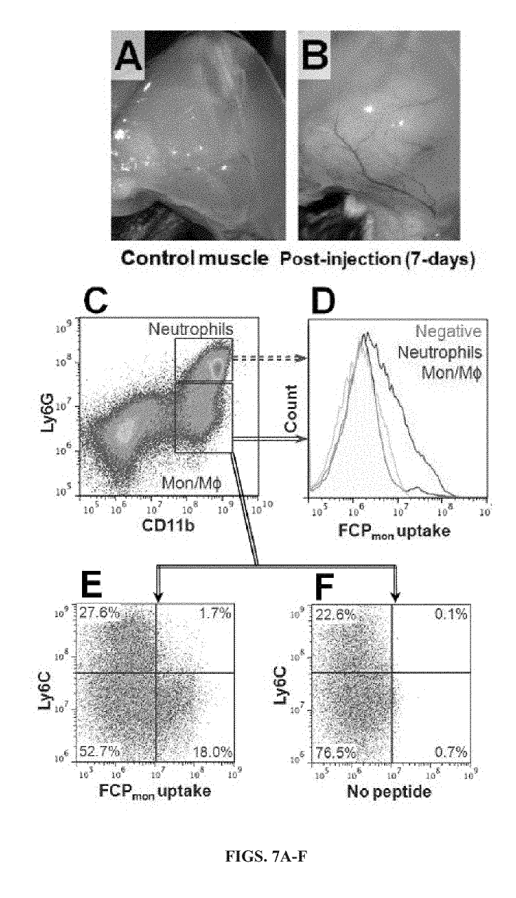

In preparation for microSPECT imaging and to test the potential in vivo detectability of FCPₘₒₙ, its nano-molar affinity to thioglycollate-elicited macrophages was confirmed (FIG. 6A). In addition, a progressive increase in mean fluorescent intensity (MFI) of cells during incubation with FCPₘₒₙ at 37° C. was observed compared to 4° C. (reaching 3.8-fold after 30 min), suggesting that active internalization of the peptide contributes to signal amplification (FIG. 6B). The specificity of FCPₘₒₙ was also confirmed by coincubation with excess unlabeled folic acid, which blocked 94% of the peptide uptake (FIG. 6B). Importantly, co-incubation with 2% fetal bovine serum (FBS) or bovine serum albumin (BSA) had no effect on the uptake of FCPₘₒₙ (FIG. 6), hence excluding a major interference from binding of FCPₘₒₙ to albumin or other plasma proteins.

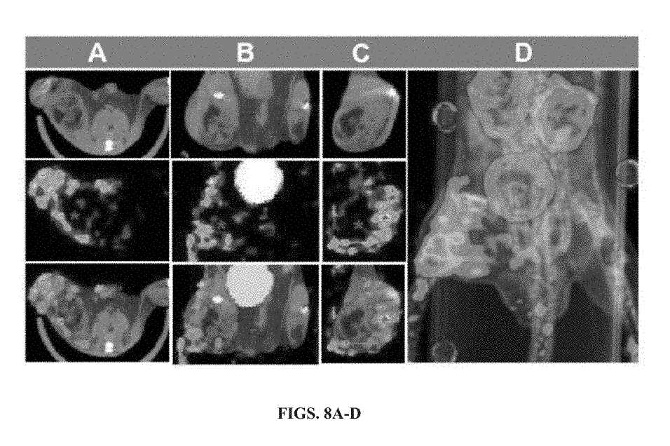

99 m-Tc labeling of FCPₘₒₙ was successfully performed by 30 min incubation of the peptide (5 nmol) with 99 m-Tc pertechnetate (5 mCi) in Tin-tricine buffer at 37° C. (Blankenberg et al., 2006). This protocol yielded more than 90% labeling efficiency at the specific activity of about 1 Ci/μmol peptide, as determined by silica gel thin layer chromatography (TLC) using different solvents. As a first step to in vivo imaging, the imaging approach was optimized in an intense focal inflammatory model induced by injection of turpentine oil (40 μl, Sigma) into thigh muscles of C57BL/6 mice (Holland et al., 2012, Levashova et al., 2009, Pellegrino 2005, Seo et al., 2010, Gowrishankar et al., 2014, Wu et al., 2014 and Autio et al., 2011). Flow cytometry of the inflamed muscles showed a progressive increase in the number of infiltrating monocytes and macrophages through a 1 week course (FIG. 7), which was hence selected as the time point of in vivo imaging. Consistent with the previous results, only monocytes and macrophages, but not other infiltrated cells, showed a significant FCPₘₒₙ uptake (FIG. 7). Interestingly, only 5.7% of CD11b³⁰/Ly6Cⁱ monocytes internalized FCP^{mon}; while, 25.4% of CD11b⁺/Ly6C⁻macrophages internalized FCP^{mon} (FIG. 7). These findings confirms that FRβ expression occurs primarily after differentiation of monocytes to macrophages as characterized by the loss of Ly6C expression.

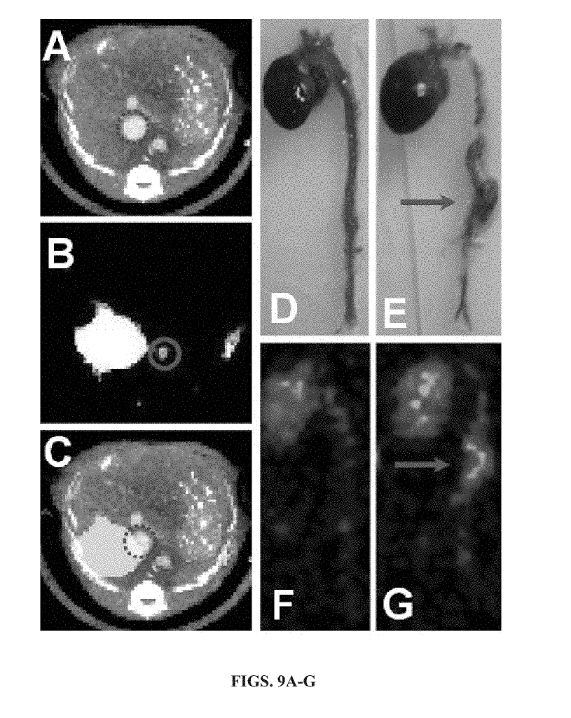

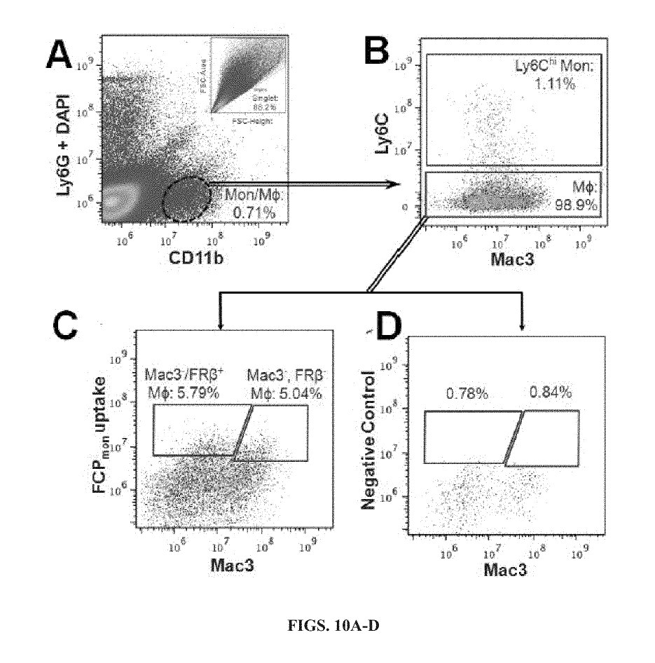

In vivo microSPECT/CT after intravenous injection of 0.5 mCi of 99 m-Tc-labeled FCPₘₒₙ shows intense focal tracer uptake in the inflammatory region which surrounds the area of oil accumulation (fat attenuation area marked by red asterisks, FIG. 8). Tracer uptake has been confirmed by a 3.3±0.4-fold increase in weight-corrected activity of the inflamed versus contra-lateral muscles by γ-counting (P<0.001). In addition, in vivo and ex vivo experiments were performed in AT-II induced AA model, which have revealed a marked increase in FCPₘₒₙ uptake in the aneurysmal segment of the aorta (FIG. 9). Together, these data strongly support the feasibility of in vivo imaging of inflammation using FCPₘₒₙ in both models of muscle inflammation and AT-II induced AA.

### Prophetic Example 2

### Spatiotemporal Distribution and Inflammatory Profile of Macrophages

It was shown that FRβ expression per se is not linked to the activation state of macrophages. Instead, it was hypothesized that FRβ expression is related to recent monocyte to macrophage differentiation. Thus, FRβ targeted imaging can be utilized to detect the ongoing influx of monocyte-derived macrophages. The dynamic relationship between FRβ expression and monocyte recruitment into inflamed peritoneum and AA will be determined. In addition, the spatiotemporal pattern of FRβ-expressing macrophages and their immunophenotypic characteristics will be determined during different stages of AA progression.

In vivo tracking of monocyte recruitment to inflamed peritoneum. An adoptive monocyte transfer technique (Nguyen et al., 2012) will be utilized in conjunction with intraperitoneal injection of 1.5 mL of 3% thioglycollate (Xia et al., 2009, Ghosn et al., 2010, Nguyen et al., 2012, Gomez et al., 2012 and Tang et al., 2011) to determine the temporal relationship between the differentiation of monocyte-derived macrophage and expression of FRβ. 1×10⁵ monocytes (approximately 2 mL of pooled mouse blood, ≈50 monocytes/μL) from CD45.1 donor mice will be enriched using a negative selection monocyte enrichment kit (Stem Cell™) and injected through the tail vein of CD45.2 recipient mice at days 0, +2 and +4 after thioglycollate injection. The enrichment efficiency (expected >90%) and their retention in circulation will be confirmed prior to and every 2 days after monocyte transfer by flow cytometry (MoFlo® Astrios). Elicited peritoneal cells will be analyzed by flow cytometry on day +6. FRβ expression and FCPₘₒₙ uptake will be determined in monocytes, SPM and LPM after separate gating on CD45.1, donor and CD45.2₊ recipient cells using CD45.1, CD45.2, CD115, Ly6C, Ly6G, F4/80 and Mac3 antibodies. Studying different time points allows discrimination of CD45.1⁺ donor monocytes-derived macrophages based on their approximate recruitment time to peritoneum, i.e., monocytes injected later (day +4) will mostly contribute to monocytes and SPMs; while those transferred earlier (day 0) will have sufficient time to reconstitute the LPM pool.

Spatiotemporal pattern of FRβ expressing macrophages and characterization of their inflammatory profile in murine AA. AA will be induced by infusion of AT-II (1000 μg/kg per day, up to 4 weeks) through subcutaneously implanted osmotic-pumps (Alzet) in 3-4 months old apoE^{-/-} mice fed with normal chow (Golestani et al., 2015, Satoh et al., 2009, Lindsay and Dietz, 2011, Daugherty and Cassis, 2004 and Bruemmer et al., 2003). Following this protocol, 73% and 26% of mice develop AA and spontaneous rupture over a 4-week period, respectively (Golestani et al., 2015). At 1, 2, and 4 weeks after AT-II infusion, aortas will be harvested, segmented based on anatomic landmarks (ascending, arch, proximal/distal thoracic, and suprarenal/infrarenal abdominal), and embedded in optimum cutting temperature (OCT). Hematoxylin & eosin (H&E) and Movat pentachrome staining will be performed for histomorphometrical measurement of total vessel area, lumen, intima, media and adventitia (NIH ImageJ software). AA will be defined as >50% increased external vessel diameter compared to adjacent segments (Kanematsu et al., 2010). The extent of inflammatory cell recruitment in different segments will be quantified in 10 sections (5-μm thickness, 1-mm intervals) by immunostaining using CD45 (pan-leukocyte marker), CD68, F4/80 and Mac3 (monocyte-macrophage), Ly6G (neutrophil), CD3 (T-cell), and CD45R/B220 (B cell) antibodies. Co-localization studies will be performed using FRβ antibody (Pierce), CD31 (endothelial cells), α-actin (vascular smooth muscle cell) and the above markers to confirm the macrophages-specificity of FRβ expression.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Aggarwal et al., Exp Clin Cardiol, 16(1):11-15, 2011.
Aggarwal et al., Experimental and Clinical Cardiology, 16(1):11-5, 2011.
Autio et al., EJNMMI Research, 1(1):10, 2011.
Ayala-Lopez et al., Journal of nuclear medicine:official publication, Society of Nuclear Medicine, 51(5):768-74, 2010.
BIOCONJUGATE TECHNIQUES (Academic Press; 1st edition, Greg T. Hermanson, 1996).
Blankenberg et al., Nat Protoc., 1(1):108-10, 2006.
Brewster DC et al., J Vasc Surg. 37:1106-17, 2003.
Brady et al., Br J Surg., 87(6):742-9, 2000.
Bruemmer et al., The Journal of Clinical Investigation, 112(9):1318-31, 2003.
Buxton, Circulation Cardiovascular Imaging., 5(3):392-9, 2012.
Chervu et al., Surgery, 117:454-7, 1995.
Courtois et al., Journal of nuclear medicine:official publication, Society of Nuclear Medicine, 2013.
Daugherty and Cassis, Arteriosclerosis, Thrombosis and Vascular Biology, 24(3):429-34, 2004.
Davies, Circulation, 98(3):193-5, 1998.
de Waard et al., Atherosclerosis, 211(1):84-9, 2010.
Dillavou et al., Journal of Vascular Surgery., 43(3):446-51, 2006.
El-Hamamsy and Yacoub, Nature reviews Cardiology, 6(12):771-86, 2009.
Ernst, N Engl J Med., 328(16):1167-72, 1993.
Farooq et al., Surgery, 119(1):9-14, 1996.
Feng et al., Arthritis Research & Therapy, 13(2):R59, 2011.
Gadowski et al., J Vasc Surg. 19:727-31, 1994.
Gadowski et al., J Vasc Surg. 19:727-31, 1994.
Gennaro, Remington: The Science and Practice of Pharmacy, Lippincott Williams & Wilkins

Publishers; 21st Ed, 2005.
Ghosn et al., Proceedings of the National Academy of Sciences of the United States of America, 107(6):2568-73, 2010.
Golestani et al., Circulation Cardiovascular imaging., 8(1), 2015.
Golledge J et al., Arterioscler Thromb Vasc Biol. 30(6):1075-1077, 2010.
Gomez et al., The Journal of Biological Chemistry, 287(7):4581-9, 2012.
Gowrishankar et al., PloS One, 9(9):e107951, 2014.
Guirguis and Barber, Am J Surg., 162(5):481-3, 1991.
Hattori et al., Journal of Liposome Research, 1-10, 2014.
Hirsch et al., Circulation. 113:e463-e654, 2006.
Hirsch et al., Circulation., 113(11):e463-654, 2006.
Holland et al., Nature Medicine, 18(10):1586-91, 2012.
Jager et al., Society of Nuclear Medicine, 55(12):1945-51, 2014.
Jiang et al., Proceedings of the National Academy of Sciences of the United States of America, 101(51):17867-72, 2004.
Kanematsu et al., Hypertension, 55(5):1267-74, 2010.
Kanematsu et al., Stroke: A Journal of Cerebral Circulation, 42(1):173-8, 2011.
Kent et al., Journal of Vascular Surgery, 52(3):539-48, 2010.
Kitagawa et al., Circulation Cardiovascular Imaging, 2013.
Kitagawa et al., Mol Imaging Biol., 14(3):315-24, 2012.
Klink et al., Nature Reviews Cardiology., 8(6):338-47, 2011.
Kotze et al., Eur J Nucl Med Mol Imaging, 38(8):1493-9, 2011.
Kudo et al., Surg Today, 34(12):1010-3, 2004.
Kuivaniemi et al., Circulation., 117(2):242-52, 2008.
Lederle et al., Archives of Internal Medicine, 160(10):1425-30, 2000.
Lee et al., Journal of Vascular Surgery, 39(3):491-6, 2004.
Lee, Methods in molecular medicine, 25:69-76, 2000.
Levashova et al., Society of Nuclear Medicine, 50(12):2058-63, 2009.
Libby et al., Journal of nuclear medicine: official publication, Society of Nuclear Medicine, 51 Suppl 1:33S-7S, 2010.
Lindsay and Dietz, Nature, 473(7347):308-16, 2011.
Lu et al., Arthritis research & Therapy, 13(2):R56, 2011.
Marini et al., Eur J Nucl Med Mol Imaging, 39(1):91-101, 2012.
McPhee et al., Journal of Vascular Surgery, 45(5):891-9, 2007.
Moehle et al., The Journal of Thoracic and Cardiovascular Surgery, 142(6):1567-74, 2011.
Nagai et al., Cancer Immunology, Immunotherapy: CII, 58(10):1577-86, 2009.
Nahrendorf et al., Arteriosclerosis, Thrombosis and Vascular Biology, 31(4):750-7, 2011.
Nevitt MP et al., N Engl J Med. 321:1009-14, 1989.
Nguyen et al., J Immunol., 189(6):3112-20, 2012.
Norman and Powell, Circulation, 115(22):2865-9, 2007.
Paulos et al., Advanced Drug Delivery Reviews, 56(8):1205-17, 2004.
Paulos et al., Arthritis Research & Therapy, 8(3):R77, 2006.
Pellegrino, Society of Nuclear Medicine, 46(9):1522-30, 2005.
Peppelenbosch et al., Eur J Vasc Endovasc Surg., 26(3):303-10, 2003.
Peppelenbosch et al., Journal of Vascular Surgery, 42(4):608-14, 2005.
Powell and Greenhalgh, N Engl J Med., 348(19):1895-901, 2003.
Puig-Kroger et al., Cancer Research, 69(24):9395-403, 2009.
Razavian et al., Society of Nuclear Medicine., 51(7):1107-15, 2010.
Richards et al., Circulation Cardiovascular Imaging, 4(3):274-81, 2011.
Satoh et al., Nature Medicine, 15(6):649-56, 2009.
Scott et al., Br J Surg. 82:1066-70, 1995.
Seibert et al., Front Neurol, 2:45, 2011.
Seo et al., Journal of Nuclear Medicine:official publication, Society of Nuclear Medicine, 51(10):1637-43, 2010.
Shen et al., Molecular Pharmaceutics, 10(5):1918-27, 2013.
Singh et al., Am J Epidemiol., 154(3):236-44, 2001.
Singh et al., Am J Epidemiol. 154:236-44, 2001.
Tang et al., Blood, 118(3):786-94, 2011.
Tavakoli, Current Cardiovascular Imaging Reports, 2:9, 2009.
Thomas and Stewart, Br J Surg., 75(8):733-6, 1988.
Turk et al., Arthritis and Rheumatism, 46(7):1947-55, 2002.
U.S. Patent Application Publication No. 2004/0242582
U.S. Patent Application Publication No. 2005/0227985
U.S. Patent Application Pulbication No. 2007/0041904
U.S. Pat. No. 8,399,403
U.S. Pat. No. 8,664,407
U.S. Pat. No. 8,778,303
Upchurch and Schaub, American Family Physician, 73(7):1198-204, 2006.
US Preventive Services Task Force Guide to Clinical Preventive Services. 2nd edn. Baltimore: Williams & Wilkins; p. 67, 1996.
Vammen et al., Br J Surg. 88:1066-72, 2001.
van der Heijden et al., Arthritis and Rheumatism., 60(1):12-21, 2009.
Varghese et al., Molecular Pharmaceutics, 4(5):679-85, 2007.
Visse et al., Circ. Res. 92:827-839, 2003.
Wilmink et al., Journal of Vascular Surgery, 30(2):203-8, 1999.
Wu et al., Theranostics, 4(5):546-55, 2014.
Xia et al., Blood., 113(2):438-46, 2009.
Yao et al., PloS one, 7(3):e33523, 2012.
Zhang et al., Anal Biochem., 332(1):168-77, 2004.

What is claimed:

1. A multimeric compound comprising at least two folate conjugated peptides (FCPs), wherein a FCP comprises a folate receptor beta (FRμ) ligand and a detectable label.

2. The multimeric compound of claim 1, wherein a first FCP of the at least two FCPs is identical to a second FCP.

3. The multimeric compound of claim 1, wherein a first FCP of the at least two FCPs is not identical to a second FCP.

4. The multimeric compound of claim 3, wherein the second FCP comprises a different FRβ ligand and/or detectable label as compared to the first FCP.

5. The multimeric compound of claim 2 or 3, wherein the FRP ligand is pteroyl-γ-glutamate or an analog thereof.

6. The multimeric compound of claim 2 or 3, wherein the detectable label comprises a chromophore.

7. The multimeric compound of claim 6, wherein the chromophore is a fluorophore.

8. The multimeric compound of claim 7, wherein the fluorophore is cyanine, fluorescein, rhodamine, DyLight Fluor or Alexa Flour.

9. The multimeric compound of claim 8, wherein the rhodamine is tetramethylrhodamine (TAMRA).

10. The multimeric compound of claim 2 or 3, wherein the detectable label is a radionuclide.

11. The multimeric compound of claim 10, wherein the radionuclide is a positron-emitting isotope or a gamma-ray isotope.

12. The multimeric compound of claim 11, wherein the gamma-ray isotope is ^{99m}Tc.

13. The multimeric compound of claim 10, wherein the radionuclide further comprises a chelating crosslinker.

14. The multimeric compound of claim 13, wherein the chelating crosslinker is hydrazinonicotinamide (HyNic), diethylene triamine pentaacetic acid (DTPA), or 1,4,7,10-tetraazacyclododecane-1,4,7,10-tetraacetic acid (DOTA).

15. The multimeric compound of claim 13, wherein the chelating crosslinker is hydrazinonicotinamide (HyNic).

16. The multimeric compound of claim 5, wherein the detectable label is conjugated to the FRβ ligand by a spacer.

17. The multimeric compound of claim 16, wherein the spacer comprises the amino acid sequence ARSK.

18. The multimeric compound of claim 16, wherein the spacer is conjugated to the γ-glutamate of the pteroyl-γ-glutamate.

19. The multimeric compound of claim 2 or 3, wherein the at least two FCPs further comprise a second detectable label.

20. The multimeric compound of claim 19, wherein the first detectable label comprises a chromophore and the second detectable label comprises a radionuclide.

21. The multimeric compound of claim 19, wherein the first detectable label comprises a radionuclide and the second detectable label comprises a chromophore.

22. The multimeric compound of claim 2 or 3, wherein the at least two FCPs are conjugated by a linker.

23. The multimeric compound of claim 22, wherein the linker is cleavable by a matrix metalloproteinase (MMP) or a cathepsin.

24. The multimeric compound of claim 23, wherein the MMP is further defined as MMP2/9.

25. The multimeric compound of claim 24, wherein the MMP2/9-cleavable linker comprises an amino acid sequence GPLGLAGRP.

26. The multimeric compound of claim 25, wherein the first FCP comprises from N-terminus to C-terminus the pteroyl-yγglutamate; a first spacer; TAMRA; a second spacer; HyNic-^{99m}Tc and the MMP2/9 cleavable linker.

27. The multimeric compound of claim 1, further comprising a therapeutic agent.

28. The multimeric compound of claim 27, wherein the therapeutic agent is a protein, a peptide, or a therapeutic nucleic acid.

29. The multimeric compound of claim 28, wherein the therapeutic nucleic acid is an antisense, a siRNA, an antisense, or a gene therapy.

30. The multimeric compound of claim 28, wherein the protein is an antibody, and antibody fragment, an scFv, or an antigen.

31. The multimeric compound of claim 27, wherein the therapeutic agent is an anti-aneurysmal agent.

32. The multimeric compound of claim 31, wherein the anti-aneurysmal agent is bisphosphonate, angiotensin-converting enzyme inhibitor, beta-blocker, or statin.

33. A pharmaceutical composition comprising a multimeric compound according to any one of claims 1-26, a therapeutic agent and an excipient.

34. The pharmaceutical composition of claim 33, wherein the pharmaceutical composition is formulated for oral, intravenous, intraarticular, parenteral, enteral, topical, subcutaneous, intramuscular, buccal, sublingual, rectal, intravaginal, intrapenile, intraocular, epidural, intracranial, or inhalational administration.

35. A method of treating an aneurysm in a subject comprising administering a therapeutically effective amount of the pharmaceutical composition of claim 33 to said subject.

36. The method of claim 35, wherein the aneurysm is an aortic aneurysm or a cerebral aneurysm.

37. A method for producing a multimeric compound comprising at least two FCPs, wherein an FCP comprises pteroyl-γ-glutamate, TAMRA, HyNic-^{99m}Tc, and a MMP2/9 cleavable linker, comprising:
(a) combining N¹⁰-(Trifluoroacetyl)pteroic acid and N-α-Fmoc-L-glutamic acid α-t-butyl ester, thereby producing pteroyl-γ-glutamate;
(b) adding Fmoc-Lysine(5-TAMRA)-OH and Fmoc-Lysine-ε-(6-Boc-HyNic);
(c) attaching the MMP2/9 cleavable linker, thereby producing an FCP; and
(d) conjugating the least two FCPs through the MMP2/9 cleavable linker, thereby producing the multimeric compound.

38. A method for detecting a rupture-prone aneurysm comprising administering an effective amount of the multimeric compound according to any one of claims 1-26 to a subject and imaging the detectable moiety, wherein focal uptake of the multimeric compound identifies a rupture-prone aneurysm.

39. The method of claim 38, wherein the subject is a human.

40. The method of claim 38, wherein the imaging comprises positron emission tomography (PET), single photon emission computed tomography (SPECT), computerized tomography (CT), or magnetic resonance imaging (MRI).

41. The method of claim 38, wherein the imaging is performed by a catheter-based device.

42. The method of claim 38, wherein the aneurysm is an aortic aneurysm or cerebral aneurysm.

43. The method of claim 42, wherein the aortic aneurysm is an abdominal aortic aneurysm or a thoracic aortic aneurysm.

44. The method of claim 42, wherein the cerebral aneurysm is a saccular aneurysm or a fusiform aneurysm.

45. The method of claim 38, wherein the subject has a previously diagnosed aneurysm.

46. The method of claim 38, wherein administering comprises injection.

47. The method of claim 46, wherein the injection is intraperioneal, intravenous or intramuscular,

48. The method of claim 38, wherein administering is performed at least 30 minutes prior to imaging.

49. The method of claim 38, wherein the MMP2/9 cleavable linker is cleaved at site of a vulnerable aneurysm.

50. The method of claim 49, wherein the multimeric compound has enhanced penetration into the vessel wall after cleavage by MMP2/9 relative to prior to cleavage.

51. The method of claim 38, wherein the multimeric compound selectively binds small peritoneal macrophages as compared to large peritoneal macrophages.

52. A method of treating a rupture-prone aneurysm comprising performing surgical repair and/or administering an anti-aneurysmal agent to the subject identified to have the rupture-prone aneurysm according to the method of claim 38.

53. The method of claim 52, wherein an anti-aneurysmal agent is bisphosphonate, angiotensin-converting enzyme inhibitor, beta-blocker, or statin.

54. The method of claim 52, wherein treating comprises inhibiting the development of or inducing the regression of the aneurysm.

55. The method of claim 52, wherein surgical repair comprises placing a stent graft.
